(19) United States
(12) Patent Application Publication
(10) Pub. No.: US 2021/0290123 A1
(43) Pub. Date: Sep. 23, 2021

(54) Device and Method of Obtaining Aseptic Blood from Fish

(71) Applicant: Sea Run Holdings, Inc., Freeport, ME (US)

(72) Inventor: Evelyn S. Sawyer, Freeport, ME (US)

(21) Appl. No.: 17/177,350

(22) Filed: Feb. 17, 2021

Related U.S. Application Data
(60) Provisional application No. 62/992,603, filed on Mar. 20, 2020.

(51) Int. Cl. A61B 5/155; A61B 5/15

(52) U.S. Cl. CPC A61B 5/155; A61B 5/150099; A61B 5/150221; A61B 2503/40; A61B 5/150343; A61B 5/150351; A61B 5/150229

(57) ABSTRACT

A method for obtaining large volumes of blood from farmed fish especially farmed salmon includes providing a container to which a negative internal pressure is applied. Input tubes to the container include valves to control the flow of fluid into the container through the input tubes. Needles at the distal ends of the tubes are used to access blood vessels of a fish. On opening of the valve associated with the tube connected to the needle accessing the fish blood vessel, the negative pressure in the container draws blood from the fish into the container. Multiple input tubes can be used to access the blood vessels of a corresponding number of fish at the same time to rapidly collect blood from an entire harvest of fish. The method preserves valuable plasma proteins, and prevents contamination of the blood.

### CROSS-REFERENCE TO RELATED APPLICATIONS

This is related to, and claims priority from, U.S. Provisional Application for Patent No. 62/992,603, which was filed on Mar. 20, 2020, the disclosure of which is incorporated herein in its entirety.

### FIELD OF THE INVENTION

The present invention relates to the finfish aquaculture (farming) industry, especially salmon farming, and the value-added products that can be obtained from the blood of farmed fish.

### BACKGROUND OF THE INVENTION

The finfish aquaculture industry is dominated by salmon farming, an industry that harvests over 2.5 million tons of fish annually. Fish blood, including salmon blood, has been viewed traditionally as a waste product for disposal. To preserve shelf life, the fish gills are cut at harvest to drain blood, but since clotting is rapid, this method is largely ineffective for collection and any collected blood is highly contaminated. Furthermore, plasma clotting factors are lost in the process. As a result, fish blood that might otherwise be put to use for research or medical applications is instead disposed of, wasting a potentially beneficial substance that could otherwise be procured as a byproduct of an existing process.

Recently salmon blood has become valuable for the many products that can be obtained from the plasma (Rothwell et aL 2005: Sharp et al. 2012; Smith et al. 2016). However, blood obtained from cutting the gills or other cuts to arteries and veins removes clotting proteins and other factors and results in contamination. Drawing blood from fish by needle and syringe or vacuum tube (Vacutainer®) can be performed for small volumes of blood (Strenge R J. 1983) and preserves plasma proteins, but multiple expensive medical-grade tubes are needed for a single fish. Furthermore, a harvest-size salmon (4.5-6.5 kg) yields only 70-100 ml of blood. Therefore, this method is impractical when large volumes of blood are required. The present invention overcomes these restraints by providing a method and device that allows for bleeding more than one fish at a time, usually three or four, by an aseptic method that preserves plasma proteins.

### BRIEF SUMMARY OF THE INVENTION

Therefore, it is an object of the present invention to obtain blood from fish, especially but not exclusively farmed salmon, in large volumes.

It is another object of the present invention to bleed more than one fish at the same time.

It is a further object of the present invention to bleed fish by aseptic methods.

It is a further object to obtain fish blood that is not depleted of plasma proteins.

According to an aspect of the invention, a method of collecting blood from fish includes providing a container partially filled with a quantity of anti-coagulant. An opening of the container is closed off with a closure that sealingly engages the opening of the container. The closure includes an aperture. A tube is provided through and sealingly engaged with the aperture, such that a proximal end of the tube is disposed inside the container above a predetermined fill level, and a distal end of the tube is disposed outside the container. Fluid communication through the tube is prevented. A pressure inside the container is maintained that is lower than the ambient pressure outside the container. Access is provided to a blood vessel of a fish at the distal end of the tube. Fluid communication is allowed through the tube, such that the lower pressure inside the container draws blood accessed at the distal end of the tube into the container through the tube, until a level of blood in the container rises no higher than the proximal end of the tube within the container. The predetermined fill level is helpful in determining an approximate and appropriate stopping point for the procedure, but the proximal end of the tube defines the point before which blood collection must stop for that container. The method can also include noticing a clot in the tube, in which case fluid communication through the tube is then prevented. Access to the blood vessel of the fish is removed at the distal end of the tube, and then fluid communication through the tube is allowed (preferably to a lesser degree than previously) and then prevented in rapid succession, allowing a brief intake of air to clear the clot. Access to the blood vessel of the fish is then again provided at the distal end of the tube to the previous degree, and fluid communication through the tube is allowed.

The method can also include continuing to draw blood from the fish into the container until blood ceases to flow in the tube due to depletion of the fish or any other reason. Fluid communication through the tube is then prevented, and access to the blood vessel of the fish at the distal end of the tube is removed. Fluid communication through the tube can be instantly allowed and prevented in order to clear blood from the tube, in a manner similar to that of clearing the clot. The pressure inside the container can be equalized with the ambient pressure outside the container and the closure can be removed from the container.

The aperture can be a plurality of apertures and the tube can be a corresponding respective plurality of tubes. In this case, providing a tube through and sealingly engaged with the aperture includes providing each tube through and sealingly engaged with a corresponding respective one of the apertures such that a proximal end of each tube is disposed inside the container, and a distal end of each tube is disposed outside the container. Fluid communication is prevented through the plurality of tubes, access is provided to the blood vessels of a plurality of fish at the corresponding respective distal ends of the plurality of tubes, and fluid communication is allowed through the plurality of tubes, such that the lower pressure inside the container draws blood accessed at the distal ends of the tubes into the container through the tubes. Fluid communication through the plurality of tubes can be allowed simultaneously.

The closure can be a stopper.

The tube can be closed off and opened using a valve.

A pump can be used to apply a partial vacuum inside the container.

Providing access to a blood vessel of a fish at the distal end of the tube can include inserting a needle coupled to the distal end of the tube into the blood vessel of the fish.

According to another aspect of the invention, a method of collecting blood from fish includes providing a container partially filled with a quantity of anti-coagulant. An opening of the container is closed off with a closure that sealingly engages the opening of the container. The closure includes a plurality of apertures. A corresponding plurality of tubes is provided through and sealingly engaged with the plurality of apertures, such that proximal ends of the tubes are disposed inside the container above a predetermined fill level, and distal ends of the tubes are disposed outside the container. Fluid communication through the tubes is prevented. A pressure is maintained inside the container that is lower than the ambient pressure outside the container. Access is provided to blood vessels of a corresponding plurality of fish at the distal ends of the plurality of tubes. Fluid communication is allowed through the tubes, such that the lower pressure inside the container draws blood accessed at the distal ends of the tubes into the container through the tubes, until a level of blood in the container rises no higher than the proximal ends of the tubes. The predetermined fill level is helpful in determining an approximate and appropriate stopping point for the procedure, but the lowest proximal end of the tubes defines the point before which blood collection must stop for that container.

The method can also include noticing a clot in one of the tubes, in which case fluid communication through that tube is then prevented. Access is removed from the blood vessel of the fish at the distal end of the tube. Fluid communication is then allowed (preferably to a lesser degree than previously) and then prevented through the tube in rapid succession, allowing a brief intake of air to clear the clot from the tube. Access is then provided to the blood vessel of the fish at the distal end of the tube, preferably to the previous degree, and then fluid communication is allowed through the tube.

Fluid communication can be prevented through any tube in which blood ceases to flow due to depletion of the fish or any other reason, and access to the blood vessel of the fish can be removed at the distal end of the tube in which blood ceases to flow. Fluid communication through the removed tube can be instantly allowed and prevented, in order to clear blood from the removed tube in a manner similar to that of clearing the clot. After blood has been cleared from all of the tubes, the pressure inside the container can be equalized with the ambient pressure outside the container and the closure can be removed from the container.

Fluid communication through the plurality of tubes can be allowed simultaneously.

According to another aspect of the invention, an apparatus for collecting blood from fish includes a container, a closure, a number of tubes, a number of valves, a pump, and a number of needles. The container is configured to be partially filled with a quantity of anti-coagulant. The closure is configured to sealingly engage the opening of the container. The closure includes a number of apertures. The corresponding number of tubes is configured to be inserted through and sealingly engaged with the number of apertures, such that proximal ends of the tubes are disposed inside the container above a predetermined fill level of the container, and distal ends of the tubes are disposed outside the container. The corresponding number of valves are coupled to the number of tubes and are configured to selectively prevent or allow fluid communication through the tubes. The pump is configured to maintain a pressure inside the container that is lower than the ambient pressure outside the container. The corresponding number of needles are coupled to the distal ends of the number of tubes and are configured to provide access to blood vessels of a corresponding number of fish at the distal ends of the number of tubes.

The apparatus can also include a selectable branched connector in at least one of the tubes. The branched connector can be configured to provide selectable fluid communication in the tube with a number of needles having different gages.

The apparatus can also include a pump tube arranged between the pump and the interior of the container, and a pump valve configured to selectively prevent or allow fluid communication through the pump tube.

### BRIEF SUMMARY OF THE DRAWING FIGURES

FIG. 1 is a schematic diagram of an exemplary apparatus that can be used to perform the method of the invention.

FIG. 2 is a flow diagram of an exemplary method of the invention.

FIG. 3 is a schematic diagram of an exemplary apparatus that can be used to perform the method of the invention.

### DETAILED DESCRIPTION OF THE INVENTION

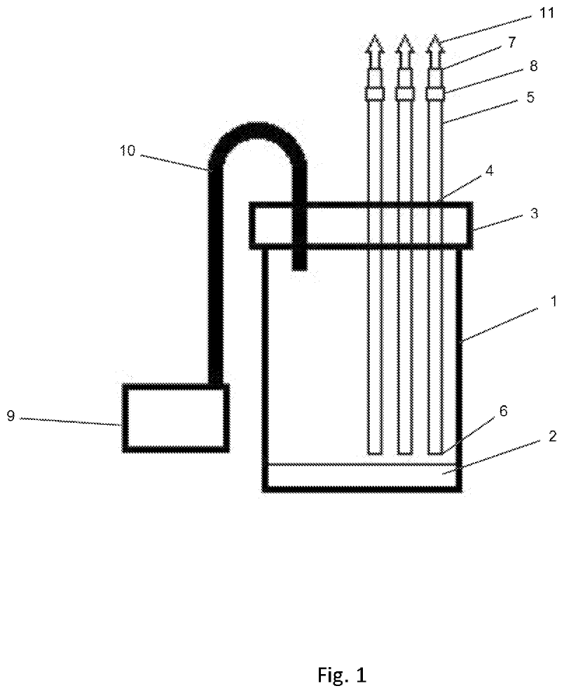

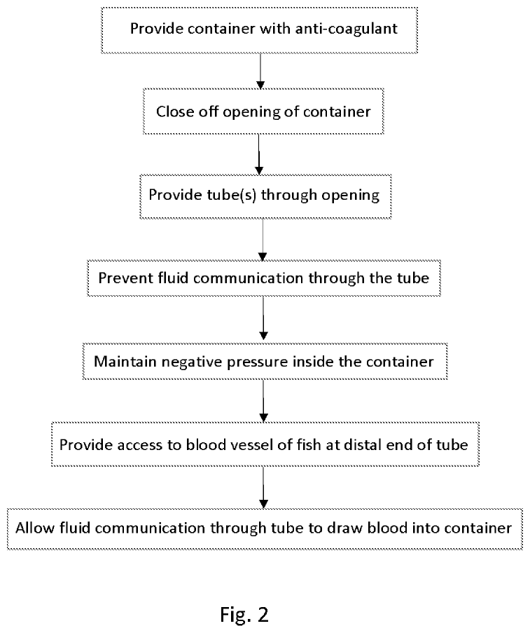

Referring to FIGS. 1 and 2, generally, the invention is a method of collecting blood from fish that includes providing a container 1 partially filled with a quantity of anti-coagulant 2. An opening of the container 1 is closed off with a closure 3, such as a cover or stopper, which sealingly engages the opening of the container 1. The closure 3 includes at least one opening 4, and preferably several openings. A bleeding tube 5 is provided through and sealingly engaged with the opening 4, such that a proximal end 6 of the bleeding tube 5 is disposed inside the container 1, and a distal end 7 of the bleeding tube 5 is disposed outside the container 1. The closure 3 can have more than one opening, in which case a bleeding tube 5 is provided for each opening 4. Fluid communication through the bleeding tube 5 is selectively prevented, such as through use of a valve 8 arranged at a location along the length of the bleeding tube 5 (or each bleeding tube) outside the container 1. Thus, the interior of the container 1 is sealed off from the environment outside the container 1. If any opening 4 is not fitted with a corresponding tube 5, that opening must be closed off in some way before the bleeding procedure begins.

A pressure inside the container 1 is maintained such that it is lower than the ambient pressure outside the container 1. This can be accomplished, for example, through the use of a pump 9, arranged inside or outside of the container 1 and able to control the pressure inside the container 1 through a pump tube 10 having a proximal end inserted through an opening in the closure. Any type of device suitable to control the relative pressure inside the container 1 with respect to ambient can be used. The pump tube 10 can include an in-line valve that can be used to control the extent of the negative pressure applied within the container 1 without accessing controls on the pump 9 itself.

Access to a blood vessel of a fish is provided at the distal end 7 of the bleeding tube 5. For example, the distal end(s) 7 of the bleeding tube(s) 5 can be fitted with a needle 11 or other implement to pierce or otherwise facilitate access to the blood vessel of the fish. Fluid communication through the bleeding tube 5 now is allowed, such as by opening the valve 8. Now, the lower pressure inside the container 1 draws blood accessed at the distal end 7 of the bleeding tube 5 into the container 1 through the bleeding tube 5. If several bleeding tubes are used, the blood of several fish can be accessed and collected in this manner, simultaneously if desired.

Because the blood is drawn into the distal end 7 of the bleeding tube 5, through the bleeding tube 5 and into the container 1 through the proximal end 6 of the bleeding tube 5, the proximal end 6 of the bleeding tube 5 should be disposed far enough away from the proximal end of the pump tube 10 such that blood drawn into the container 1 is not sucked directly into the pump tube 10. For example, the proximal end of the pump tube can be disposed near the top of the container 1, whereas the bleeding tube(s) 5 can be arranged such that the proximal ends 6 are more spaced from the top of the container 1.

It will be apparent to one of skill in the art that a container having an integrated port for the pump tube and/or one or more ports for the bleeding tube(s) can be used. The ports can be arranged at the top and/or side(s) of the container to suit the application. The port(s) can be fitted with a sealing edge such as a grommet to provide a fluid-tight seal with a tube inserted through the port.

An exemplary method of obtaining blood from farmed salmon is described according to the present invention.

An exemplary apparatus used to practice the invention includes a container such as a standard 500 ml or 1 liter flask or bottle containing anti-coagulant such as citrate or EDTA sufficient for the volume of blood to be collected, and fitted with a stopper or cover having a number of holes, such as four or five holes. Tubing from one hole extends from the top of the container to a pump with a setting of, for example, 8-12 inches of mercury (Hg) vacuum. This pump tube can include a shut-off valve, preferably in-line, to control application of the suction. The other holes have tubing extending through the hole to within a distance from the bottom of the flask. The further ends of these tubes are connected to a 2-way valve and then continue to a needle, preferably a 14-18 gage needle. Preferably all components are sterile. Initially all valves are closed, and the pump is turned on.

According to an exemplary process of the invention, at harvest directly after fish are stunned, or for anesthetized broodstock prior to spawning, the fish are placed ventral side up in racks of wire or other stiff material that holds the fish in that position. The caudal peduncle of the fish is then sprayed with ethanol. The needle from one tube is inserted centrally between the anal and caudal fins to access the caudal artery and vein. The 2-way valve on that tube is now turned on to allow blood to flow from the fish to the flask. This process is repeated for the other tubes with other fish. When blood ceases to flow in a tube, the valve is closed before the needle is removed from the fish. As each fish is drained of blood, a new fish is placed in the rack and the process is repeated.

To cope with coagulation in the tubing, the valves may be opened and shut rapidly to clear the tubing, or a syringe containing less than 5 ml or so of anticoagulant may be fitted to the needle end of the tubing and flushed through the tubing and valve into the flask.

When the flask has been adequately filled, it is closed with a solid stopper and placed in a cooler at 4-6° C. for storage or transport, such as to a laboratory for centrifugation.

This process can produce a liter of blood in 15-20 minutes from 10-15 salmon depending on size. The time depends largely on how quickly fish are replaced in the racks.

Although the method is described for farmed salmon, it could be applied to other species of farmed fish, or to wild-caught fish.

The device and method described above is effective, but can present a persistent limitation related to a problem with the formation of blood clots in the lines, that is, in the tubing that leads from the fish to the vacuum blood container (VBC).

Typical tubing material is made from silicone (Tygon®) or other clot-resistant material with, for example, an ⅛″ internal diameter. Preferably, it extends from a 16 gage or larger diameter needle in the fish through a typically plastic OFF/ON valve and into the VBC. Before use, the tubing is flushed with an anticoagulant solution. These anti-clotting measures are satisfactory for mammalian blood, but the blood of salmonids (salmon and trout) has remarkable clotting capability (Rothwell et al. 2005). Any irregularity on the inside of the tubing, or an interruption of flow such as which occurs at the valve or at connectors, frequently triggers a clot that blocks the flow of blood.

There are two conventional methods used to clear the clot, both of which are not entirely satisfactory.

The first conventional method uses an anticoagulant to remove clots. First, the valve is closed, and then the needle is removed from the fish and placed in a container of anticoagulant. The valve is then opened, and anticoagulant is drawn through the tubing into the VBC. The valve is then closed, the needle is removed from the anticoagulant container and reinserted into the fish. The valve is then opened, which causes varying amounts of anticoagulant to mix with and dilute the blood. This removes the clot, but makes the blood unusable for certain applications.

The second conventional method uses forced air to clear clots in the tubing. First, the valve is closed, and then the needle is removed from the fish. The valve is then opened and closed as quickly as possible to allow air to flush clots from the tubing. With a 16 gage (1.194 mm) internal diameter needle at the end of the tubing, it is difficult or impossible to turn the valve on and off quickly enough to limit the air entering the vacuum container. Each on/off turn of the valve allows about 85-110 cc of air into the VBC, depending on the speed of the operator. This procedure reduces the vacuum, and if a VBC is nearly full of blood, allows blood to be sucked into the line connecting the VBC to the vacuum pump, damaging the pump. When using the usual 500 ml flask as a VBC, no more than 2 of these tube-clearing procedures can typically be used when filling the flask with blood.

There is a need to limit intake air to an amount that will clear the clot(s), but will not make the vacuum ineffective. According to the invention, such a procedure begins by closing the valve, and then removing the needle from the fish. The needle is then changed to a smaller gage needle. For example, if a typical 16 gage needle is being used, it can be removed and replaced, for example, by a 25 gage (0.26 mm ID diameter) needle. Any substitution to a smaller sized needle would be beneficial. The valve can then be opened and closed as quickly as possible, allowing air into the system. With the valve still closed, the needle can be changed back to the original needle, or to another needle larger than the needle used to flush the clot. The needle is then inserted in the fish, and the valve is opened to resume drawing blood.

According to this method, it is easier to limit the amount of air used to flush the clot in order minimize the possibility of damaging the pump. That is, by restricting air flow with the smaller diameter needle, when the operator turns the valve on and off quickly, only about 30-40 cc of air is allowed into the VBC. This is enough air to clear even stubborn clots, and allows up to four tube-clearing procedures for a 500 ml VBC without reducing vacuum to an insufficient level or allowing blood to be sucked into the VBC-pump line.

The apparatus of the invention can be modified to facilitate performing the clot-clearing method. For example, the tubing near the needle can be fitted with a T-valve that normally allows flow from the needle but when switched cuts off the needle and instead allows flow from an alternative source. This source can be a piece of open narrower tubing, tubing having a smaller gage needle attached to the end, or some other more restrictive fluid intake. When this T-valve is switched between positions, air is allowed into the system to flush the clot as described above, and then switched back to the fish, providing for a shorter interruption of the bleeding process. The T-valve can be biased so that it automatically returns to the original position, and can be ratcheted to be click-advanced by the press of a button, so that the switching speed is not subject to the skill of the operator but instead is reliable within the range of operation of the biased valve. Alternatively, the T-valve, tubing, or needle can have a damper that limits the amount of intake air provided at the valve, or the intake air can be provided by a measured, pressurized source.

Another persistent problem is the difficulty in determining whether there is blood flow through the blood tubes. In order to efficiently collect blood from the donor fish, a fish that has been depleted should be quickly replaced by a fresh donor fish, and one way to determine that it is time to switch donor fish is by observing if blood flow in a tube ceases. However, tubes appear red with blood even when the blood is not flowing, and therefore flow cannot reliably be detected merely by observing the tubes. If the proximal end of the blood tube is near the bottom of the VBC as shown in FIG. 1, it is not possible to see if blood is flowing and entering the VBC because the opening is below the collected blood level. This problem has been addressed by one of three conventional methods, which are less than completely satisfactory. The first method of detecting flow is by using a flowmeter, such as an off-the-shelf device sold by medical device companies, inserted in a bleeding tube or line. It has been found however, that these devices slow the flow of blood and therefore promote clotting. In addition, the devices can cause hemolysis in salmon blood. Unlike the generally round human red blood cells, fish red blood cells vary in size and shape. The hemolysis may be caused by the more angular shape of salmon red blood cells. The second conventional method includes weighing the VBC, and noting that an increase in weight indicates blood flow. This is difficult without the time-consuming process of detaching the blood tubes from the fish or the VBC. Also, when multiple fish are being bled simultaneously, this method does not reliably indicate when blood flow in only one of the tubes has stopped, and can't indicate in which tube flow has stopped.

According to the third conventional method, the operator must closely and carefully observe the blood level rise in the VBC. This requires a clear glass VBC, instead of the usual preferred plastic material required in a fish processing environment. Glass VBCs are often not allowed in such an environment due to the danger of broken glass in a food product. Again, when multiple fish are being bled simultaneously, this method does not reliably indicate when blood flow in only one of the tubes has stopped, and can't indicate in which tube flow has stopped.

According to the invention, determination of whether blood is flowing in any tube can be made by the positioning of the blood tubes in the VBC. The VBC closure can hold the tubes such that the proximal ends of the tubes are arranged above the fill-line or desired fill level of the VBC, so that dripping of blood from tubes in which blood is flowing can be observed easily, as can lack of dripping, which indicates that flow has stopped in that tube. If desired, the tubes can be labeled or marked at the ends for easy identification.

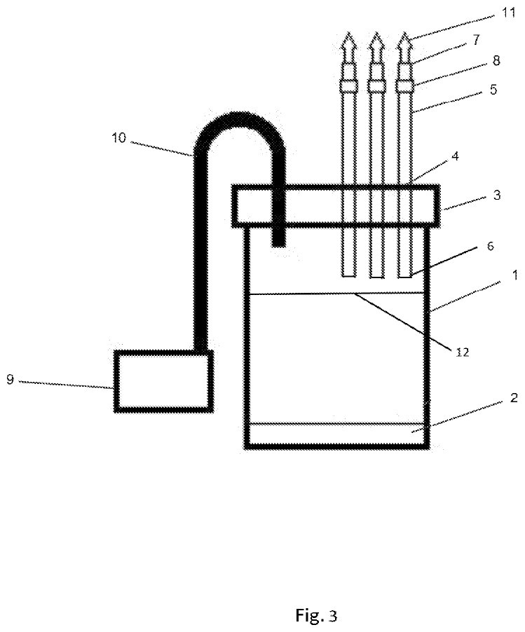

As shown in the exemplary embodiment of FIG. 3, the VBC can be a typical flask having a fill-line 12 indicating a volume of 500 ml. The tubes are held within through-holes in a plug closing the VBC such that the proximal ends 6 of the tubes are held above the fill-line 12 by some distance in which dripping blood is easily observable, for example, a half-inch above the fill-line 12. The proximal ends 6 of the blood tubes should also be arranged lower than the pump tube, for example by at least 2 inches, to prevent suction of blood into the pump. The pump tube can be firmly held in a through-hole in the plug to ensure that this separation is maintained, or the tubing and/or through-hole can include a cooperative feature, such as keying or an external bulge, to maintain the separation.

Of course, the VBC does not have to have an actual fill-line, and if it does have a fill-line this line does not necessarily determine the extent to which the VBC can or should be filled. The actual fill level can be any height regardless of any fill-line, as long as the fill level reached during the bleeding process is lower than the proximal ends of the blood tubes within the VBC. Therefore, any actual fill-line showing on the VBC can be useful as a convenience, but is not a limitation on the process of the invention. Also, if multiple bleeding tubes are used, the proximal ends of the bleeding tubes might not be arranged at the same level. In this case, the maximum fill level for the process is preferably just below the lowest proximal end of the multiple bleeding tubes.

The invention has been described by way of example and in terms of preferred embodiments. However, the present invention as contemplated by the inventor is not strictly limited to the particularly disclosed embodiments, To the contrary, various modifications, as well as similar arrangements, are included within the spirit and scope of the present invention. The scope of the appended claims, therefore, should be accorded the broadest reasonable interpretation so as to encompass all such modifications and similar arrangements.

### REFERENCES

Rothwell S W, Reid T J, Dorsey J, Flourney W S, Sawyer E S, Janmey P A. 2005. A salmon fibrinogen-thrombin bandage controls arterial bleeding in a swine aortotomy model. J. Trauma 59)1):143-149.
Sharp K G, Dickson A R, Marchenko S A, Yee K M, Emery P N, Laidmae I, Uibo R, Sawyer E S, Steward O, Flanagan L A. 2012. Salmon fibrin treatment of spinal cord injury promotes functional recovery and density of serotonergic innervation. Exp Neurol. 235(1):345-56.
Smith J R, Galie P A, Slowchower D R, Weisshaar C L, Janmey P A, Winkelstein B A. 2016. Salmon thrombin inhibits chronic pain through an endothelial barrier protective mechanism dependent on APC. Biomaterials 80 Febuary: 96-105.
Strange R J. 1983 in Fisheries Techniques. Nelsen L A and Johnson D L (Ed). pp 345-346.

I claim:

1. A method of collecting blood from fish, comprising:
providing a container partially filled with a quantity of anti-coagulant;
closing off an opening of the container with a closure that seal ingly engages the opening of the container, wherein the closure includes an aperture;
providing a tube through and sealingly engaged with the aperture, such that a proximal end of the tube is disposed inside the container above a predetermined fill level, and a distal end of the tube is disposed outside the container;
preventing fluid communication through the tube;
maintaining a pressure inside the container that is lower than the ambient pressure outside the container;
providing access to a blood vessel of a fish at the distal end of the tube; and
allowing fluid communication through the tube, such that the lower pressure inside the container draws blood accessed at the distal end of the tube into the container through the tube, until a level of blood in the container rises no higher than the proximal end of the tube.

2. The method of claim 1, further comprising:
noticing a clot in the tube;
preventing fluid communication through the tube;
removing access to the blood vessel of the fish at the distal end of the tube;
allowing, to a lesser degree than previously, and then preventing fluid communication through the tube in rapid succession, thereby clearing the clot from the tube;
providing access to the blood vessel of the fish at the distal end of the tube to the previous degree; and
allowing fluid communication through the tube.

3. The method of claim 1, further comprising:
continuing to draw blood from the fish into the container until blood ceases to flow in the tube;
preventing fluid communication through the tube; and
removing access to the blood vessel of the fish at the distal end of the tube.

4. The method of claim 3, further comprising rapidly in succession allowing and preventing fluid communication through the tube, thereby clearing blood from the tube.

5. The method of claim 3, further comprising:
equalizing the pressure inside the container with the ambient pressure outside the container; and
removing the closure from the container.

6. The method of claim 1, wherein:
the aperture is a plurality of apertures;
the tube is a corresponding respective plurality of tubes; and
providing a tube through and sealingly engaged with the aperture, includes providing each said tube through and sealingly engaged with a corresponding respective one of said apertures such that a proximal end of each said tube is disposed inside the container, and a distal end of each said tube is disposed outside the container.

7. The method of claim 6, wherein;
preventing fluid communication through the tube includes preventing fluid communication through the plurality of tubes;
providing access to a blood vessel of a fish at the distal end of the tube includes providing access to the blood vessels of a plurality of fish at the corresponding respective distal ends of the plurality of tubes; and
allowing fluid communication through the tube includes allowing fluid communication through the plurality of tubes, such that the lower pressure inside the container draws blood accessed at the distal ends of the tubes into the container through the tubes.

8. The method of claim 7, wherein allowing fluid communication through the plurality of tubes includes allowing simultaneous fluid communication through the plurality of tubes.

9. The method of claim 1, wherein the closure is a stopper.

10. The method of claim 1, wherein preventing fluid communication through the tube includes closing off the tube with a valve.

11. The method of claim 1, wherein maintaining a pressure inside the container that is lower than the ambient pressure outside the container includes using a pump to apply a partial vacuum inside the container.

12. The method of claim 1, wherein providing access to a blood vessel of a fish at the distal end of the tube includes inserting a needle coupled to the distal end of the tube into the blood vessel of the fish.

13. The method of claim 1, wherein allowing fluid communication through the tube includes opening the tube with a valve.

14. A method of collecting blood from fish, comprising:
providing a container partially filled with a quantity of anti-coagulant;
closing off an opening of the container with a closure that sealingly engages the opening of the container, wherein the closure includes a plurality of apertures;
providing a corresponding plurality of tubes through and sealingly engaged with the plurality of apertures, such that proximal ends of the tubes are disposed inside the container above a predetermined fill level, and distal ends of the tubes are disposed outside the container;
preventing fluid communication through the tubes;
maintaining a pressure inside the container that is lower than the ambient pressure outside the container;
providing access to blood vessels of a corresponding plurality of fish at the distal ends of the plurality of tubes; and
allowing fluid communication through the tubes, such that the lower pressure inside the container draws blood accessed at the distal ends of the tubes into the container through the tubes, until a level of blood in the container rises no higher than the proximal ends of the tubes.

15. The method of claim 14, further comprising:
noticing a clot in one of the tubes;
preventing fluid communication through the one of the tubes;
removing access from the blood vessel of the fish at the distal end of the one of the tubes;
allowing, to a lesser degree than previously, and then preventing fluid communication through the one of the tubes in rapid succession, thereby clearing the clot from the one of the tubes;
providing access to the blood vessel of the fish at the distal end of the one of the tubes to the previous degree; and
allowing fluid communication through the one of the tubes.

16. The method of claim 14, further comprising:
preventing fluid communication through any tube in which blood ceases to flow; and
removing access to the blood vessel of the fish at the distal end of the tube in which blood ceases to flow.

17. The method of claim 16, further comprising rapidly in succession allowing and preventing fluid communication through the removed tube, thereby clearing blood from the removed tube.

18. The method of claim 17, further comprising:
after blood has been cleared from all of the tubes, equalizing the pressure inside the container with the ambient pressure outside the container; and
removing the closure from the container.

19. The method of claim 14, wherein allowing fluid communication through the plurality of tubes includes allowing simultaneous fluid communication through the plurality of tubes.

20. Apparatus for collecting blood from fish, comprising;
a container partially configured to be filled with a quantity of anti-coagulant;
a closure configured to sealingly engage the opening of the container, wherein the closure includes a plurality of apertures;
a corresponding plurality of bleeding tubes configured to be inserted through and sealingly engaged with the plurality of apertures, such that proximal ends of the bleeding tubes are disposed inside the container above a predetermined fill level of the container, and distal ends of the bleeding tubes are disposed outside the container;
a corresponding plurality of bleeding valves couple to the plurality of bleeding tubes and configured to selectively prevent or allow fluid communication through the bleeding tubes;
a pump configured to maintain a pressure inside the container that is lower than the ambient pressure outside the container; and
a corresponding plurality of needles coupled to the distal ends of the plurality of bleeding tubes and configured to provide access to blood vessels of a corresponding plurality of fish at the distal ends of the plurality of bleeding tubes.

21. The apparatus of claim 20, further comprising a selectable branched connector in at least one of the bleeding tubes, wherein the branched connector is configured to provide selectable fluid communication in the bleeding tube with a plurality of needles having different gages.

22. The apparatus of claim 20, further comprising:
a pump tube arranged between the pump and the interior of the container; and
a pump valve configured to selectively prevent or allow fluid communication through the pump tube.
